SEQUENCE LISTING (6 sequences)

SEQ ID NO: 1 - DNA, 24 bp, Artificial Sequence
  probe
gcgaggttcatgtttgtttatttc

SEQ ID NO: 2 - DNA, 19 bp, Artificial Sequence
  probe
actggccgttagcatttca

SEQ ID NO: 3 - DNA, 21 bp, Artificial Sequence
  probe
gccggagcggtcgagttctgg

SEQ ID NO: 4 - DNA, 22 bp, Artificial Sequence
  probe
ggaggcgtcccggaagttcgtg

SEQ ID NO: 5 - DNA, 21 bp, Artificial Sequence
  probe
gactggttccaattgacaagc

SEQ ID NO: 6 - DNA, 21 bp, Artificial Sequence
  probe
gcaaatggcattctgacatcc

(19) United States
(12) Patent Application Publication
(10) Pub. No.: US 2002/0164613 A1
(43) Pub. Date: Nov. 7, 2002

(54) Method of determining gene copy number

(76) Inventors: Lorelie H. Villarete, Alameda, CA (US); Mandeep Ghuman, Huntington Beach, CA (US); Wayne W. Li, Poway, CA (US)

Correspondence Address: PERKINS COIE LLP, P.O. BOX 2168, MENLO PARK, CA, 94026, US

(21) Appl. No.: 10/010,071

(22) Filed: Nov. 6, 2001

Related U.S. Application Data
(60) Provisional application No. 60/246,265, filed on Nov. 6, 2000.

(51) Int. Cl. C12Q 1/68; C12P 19/34; C12N 15/87

(52) U.S. Cl. 435/6; 435/455; 435/91.2

(57) ABSTRACT

A method for quantitatively determining the relative copy number of a gene of interest introduced recombinantly into a cell is disclosed. The method includes the steps of propagating, in a suitable culture medium, cells that are transformed with a chimeric gene construct containing and capable of expressing the gene introduced into the cells, where said construct includes (i) a control nucleic acid segment having a region of DNA capable of hybridizing with a region of native genomic DNA associated with the cell; and (ii) a second nucleic acid region that contains said gene of interest; isolating DNA from the cells; and determining the ratio of the control nucleic acid segment to the region of native genomic DNA in the isolated DNA as a quantitative measure of the relative copy number of the gene in the cell.

### FIELD OF THE INVENTION

The present invention relates to a method of quantitatively determining the relative amount of a gene of interest in a cell. In particular, the invention includes a method of determining the number of copies of a gene in Pichia pastoris.

### REFERENCES

Ausubel, F. M., et al. (1994) Current Protocols in Molecular Biology (New York: Greene Publishing Associates and Wiley-lnterscience).

Bacila et al. eds. (1978) Biochemistry and Genetics of Yeast.

Botstein et al. (1979) Gene 8:17-24.

Brake et al. (1984) Proc. Natl. Acad. Sci. USRA 81:4642-4646.

Calmels, et al. (1991) Curr. Genet. 20, 309-314.

Cohen et al. (1980) Proc. Natl. Acad. Sci. USA 77:1078.

Henikoff et al. (1981) Nature 283:835.

Higgins, D. R., and Cregg, J. M. (1998) Pichia Protocols. In Methods in Molecular Biology, Vol. 103. (J. M. Walker, ed. Humana Press, Totowa, N.J.).

Hollenberg et al. (1981) Curr. Topics Microbiol. Immunol. 96:119.

Innis et al., (1989) PCR Protocols, Academic Press, N.Y.

Mercereau-Puigalon et al. (1980) Gene 11: 163.

Mulsant, P. et al. (1988) Phleomycin Resistance as a Dominant Selectable Marker in CHO Cells. Somat. Cell Mol. Genet 14, 243-252.

Panthier et al. (1980) Curr. Genet. 2:109.

Perez et al. (1989). Plant Mol. Biol. 13, 365-373.

Rose and Harrison, eds. (1987) The Yeasts (2^{nd} ed.).

Sambrook et al. (1989) Molecular Cloning. A Laboratory Manual (Cold Spring Harbor Laboratory Press, Plainview, N.Y.).

Skinner et al., eds. (1980) Biology and Activities of Yeast (Soc. App. Bacteriol. Symp. Series No. 9).

Southern, E. M. (1975), J. Mol. Biol., 98:503.

### BACKGROUND OF THE INVENTION

Nucleic acids carry within their sequence the hereditary information and are therefore the prime molecules of life. Nucleic acids are found in all living organisms including bacteria, fungi, viruses, plants and animals. It is of interest to determine the relative abundance of nucleic acids in different cells, tissues and organisms over time under various conditions, treatments and regimes.

Increased plasmid stability and copy number are important to the biotechnology industry as a means of maintaining the production of plasmid-encoded proteins at a consistently high level. The technical hurdles associated with the quantitative determination of relative amounts of nucleic acids are daunting. Often, only a few copies of a particular nucleic acid may be present within complex mixtures. Furthermore, the availability of sample genomic DNA may be rather limited. A highly sensitive method is required to detect and determine the number of copies of a particular gene in a transformed cell.

Pichia pastoris is a methylotrophic yeast that has become a highly successful eukaryotic system for the expression of heterologous genes. It is capable of metabolizing methanol as its sole carbon source by inducing the production of alcohol oxidase. Although P. pastoris codes for two alcohol oxidase genes, AOX1 and AOX2, the AOX1 gene is responsible for 85% of the alcohol oxidase activity in the yeast cell. In addition, the use of secretion signal sequences such as the Saccharomyces cerevisiae α factor prepro peptide or the PHO1 signal have been successful in the secreted expression of heterologous protein in the P. pastoris system.

Various expression vectors for the P. pastoris system are commercially available from Invitrogen (Carlsbad, Calif.). Among them is the pPICZ series that contain the Sh ble gene from Streptoalloteichus hindustanus that confers resistance to the drug Zeocin™. This gene has been shown to efficiently confer Zeocin™ resistance in both E. coli (driven by the EM7 promoter) and P. pastoris (by the TEF1 promoter). The pPICZ plasmid series also contains the AOX1 promoter, transcriptional termination sequence from P. pastoris, as well as sequences required for plasmid replication and maintenance in bacteria.

Since P. pastoris has no stable episomal vectors, these vectors, once linearized, generate stable transformants of P. pastoris via homologous recombination between sequences shared by the vector and host genome, such as the AOX1 5′ and 3′ sequences. Such integrants show extreme stability in the absence of selective pressure even when present as multiple copies. Multiple insertion events at a single locus occur spontaneously with a frequency of 1-10% of the transformants. The generation of recombinant strains with multiple copies of the expression plasmid integrated into the genome has been shown to result in an increase in heterologous protein production via a gene dosage effect for a number of different heterologous genes. Thus, determination of clones with multiple copies of the vector insert using DNA analysis methods leads to a high probability of increasing the expression yields of the desired protein.

### SUMMARY OF THE INVENTION

A non-radioactive method using chemiluminescence detection for determining gene copy number in P. pastoris recombinants transformed with pPICZ-derived expression vectors is described.

Accordingly, it is an object of the invention to quantitatively determining the relative copy number of a gene of interest introduced recombinantly into a cell; including the steps of propagating, in a suitable culture medium, cells that are transformed with a chimeric gene construct containing and capable of expressing the gene introduced into the cells, where the construct includes a control nucleic acid segment having a region of DNA capable of hybridizing with a region of native genomic DNA associated with the cell; and a second nucleic acid region that contains the gene of interest; isolating DNA from the cells; and determining the ratio of the control nucleic acid segment to the region of native genomic DNA in the isolated DNA as a quantitative measure of the relative copy number of the gene in the cell.

It is another object of the invention to determin the ratio using the steps of; separating the isolated DNA; hybridizing a labeled nucleic acid probe to the control nucleic acid segment and to the region of native genomic DNA; and comparing signal intensity of the label hybridized to the control nucleic acid segment with the region of native genomic DNA.

In one aspect, the determining step includes amplifying the control nucleic acid segment and the region of native genomic DNA to generate two PCR products and comparing signal intensity of the PCR products. In a related aspect the amplification is performed using labeled PCR primers. The PCR products can be separated and a labeled nucleic acid probe can be hybridized to the products.

In addition, the chimeric gene construct can be integrated into the cell genome, and determination of the ratio is used to confirm stability of the gene of interest.

In one embodiment, the method can detect less than or equal to 10 picograms of said control nucleic acid segment or said region of native genomic DNA. In another embodiment, the method can detect less than or equal to 100 picograms of said control nucleic acid segment or said region of native genomic DNA.

In one embodiment, determining the ratio includes the steps of transferring the isolated DNA to first and second regions on a substrate; on the first substrate region, hybridizing a first labeled nucleic acid probe to a region of DNA present in the genomic DNA, but not present in the construct; on the second substrate region, hybridizing a second labeled nucleic acid probe to a region of DNA present in the construct, but not present in the genomic DNA; and comparing signal intensity of the first and second labeled probes.

In another embodiment, the isolated DNA is transferred to the first and second substrate regions in equal quantities. In yet another embodiment, the substrate is a nylon membrane.

In another embodiment the cells are yeast cells. In a related embodiment the cells are P. pastoris cells. In an additional embodiment the label is digoxigenin. In one embodiment, non-radioactive probes labeled with Digoxigenin-11-dUTP using PCR were produced.

These and other objects and features of the invention will be more fully appreciated when the following detailed description of the invention is read in conjunction with the accompanying drawings.

### BRIEF DESCRIPTION OF THE DRAWINGS

FIG. 1A shows a map of the pPICZ vector showing the relative location of the vector-specific digoxigenin-labeled probes. The three probes were designed to bind to specific regions within the pPICZα or Pepgen's modified pPICZ vector. The Zeocin probe binds to the Zeocin region and the AOX1 probe binds to the AOX1 region. The NTO probe contains the PHO1 signal and gene insert along with the AOX1 region.

FIG. 1B shows an ethidium bromide stained 6% polyacrylamide electrophoresis gel of probe PCR products having various probe lengths. The Zeocin PCR product was verified by DNA sequencing but shows a band slightly lower than the expected 222 bp band length. This discrepancy may be due in part to the conformation of the PCR product when run on a 6% PAGE.

FIG. 1C is a dilution series of the probes compared to the DIG-labeled control ladder to determine the probe concentration. Using spot-densitometry analysis, the concentrations were found to be: NTO probe=210 ng/ml; AOX1 probe=27.7 ng/ml; and the Zeocin probe=35.5 ng/μl.

FIGS. 2A-2C show the sensitivity of digoxigenin-labeled probes. Serial 2 dilutions of Sac I-cut pNTO4 plasmid were loaded onto three 0.7% agarose gels and transblotted onto a nylon membrane. The blots were probed with 100 ng of the NTO4 probe (2A), the Zeocin probe (2B) or the AOX1 probe (2C). The limit of detection for all three probes was about ≦83 picograms of DNA.

FIGS. 3A-3B show the specificity of digoxigenin-labeled probes. 10 g of Sac I-cut genomic DNA from the untransformed parental Pichia pastoris strain, X-33, and recombinant clone, NTO7, were run on 0.7% agarose gels. The pNTO4 plasmid cut with Sac I was used as the positive control for all three probes. (3A) Southern blots of these gels probed with either the NTO4 or the AOX1 probes consistently showed hybridization with an approximately 10 kb band and an approximately 4 kb band from the recombinant genomic DNA. However, these two probes hybridized to only the 10 kb band from the X-33 strain. The Zeocin probe which specifically hybridizes to the pPICZα-derived vectors, only hybridized to the lower 4 kb band. (3B) Panel B shows the ethidium bromide-stained gels of the transblots. The transblot from gel #1 was probed with the NTO4 probe. The transblot from gel #2 was first probed with the Zeocin probe, then stripped and reprobed with the AOX1 probe.

FIGS. 4A-4B show that the ratios of the 10 kb and 4 kb band intensities vary between Pichia pastoris recombinants. Duplicate 0.7% agarose gels were loaded with Sac I-cut genomic DNA from X-33 strain along with three different recombinant clones. (4A) Ethidium bromide stained gels. (4B) Transblots of the gels were probed with either 100 ng of the Zeocin probe or 100 ng of the AOX1 probe. The approximately 10 kb band represents AOX1 -specific DNA and the approximately 4 kb band represent the vector-specific DNA.

FIGS. 5A-5B show that the disappearance of the Sac I sites yield a higher molecular weight vector-specific DNA band. (5A) Southern blots containing genomic DNA from the X-33 strain and the recombinant clone pPICZa #22, were probed with the AOX1, Zeocin or the NTO4 probe. All three probes hybridized to an approximately 22 kb band present in the pPICZα #22 lanes but not in the X-33 lanes. (5B) Densitometric analysis of duplicate slot blots hybridized with either the AOX1 probe or the Zeocin probe show that pPICZα#22 recombinant clone to have an estimated 6 cassette copies of vector DNA inserted into the Pichia pastoris genome. The pPICZα#22 cassette length is about 3.6 kb. Note that the pPICZα#22 DNA densitometry values from each blot was standardized to that of the plasmid pNTO4 control DNA from the same blot (e.g. pPICZα#22 value / pNTO4 value). The standardized values were then used to determine the percent AOX1 ratios that represent the percentage of the AOX1 probed intensity belonging to the pPICZα#22 vector DNA.

### DETAILED DESCRIPTION OF THE INVENTION

I. Definitions

“Recombinant host cells”, “host cells”, “cells”, “cell lines”, “cell cultures”, and other such terms denoting microorganisms or higher eukaryotic cell lines cultured as unicellular entities, are used interchangeably, and refer to cells which can be, or have been, used as recipients for recombinant vector or other transfer DNA, and include the progeny of the original cell transfected. It is understood that the progeny of a single parental cell may not necessarily be completely identical in morphology or in genomic or total DNA complement as the original parent, due to accidental or deliberate mutation. Progeny of the parental cell which are sufficiently similar to the parent to be characterized by the relevant property, such as the presence of a nucleotide sequence encoding a desired peptide, are included in the progeny intended yb this definition, and are covered by the above terms.

An “open reading frame” is a region of a polynucleotide sequence which encodes for a polypeptide.

By “yeast” is intended ascosporogenous yeasts (Endomycetales), basidiosporogenous yeasts, and yeast belonging to the Fungi Imperfecti (Blastomycetes). The ascosporogenous yeasts are divided into two families, Spermophthoraceae and Saccharomycetaceae. The later is comprised of four subfamilies, Schizosaccharomycoideae (e.g., genus Schizosaccharomyces), Nadsonioideae, Lipomycoideae, and Saccharomycoideac (e.g., genera Pichia, Kllyveromyces, and Saccharomyces). The basidiosporogenous yeasts include the genera Leucosporidium, Rhodosporidium, Sporidiobolus, Filobasidium, and Filobasidiella. Yeast belonging to the Fungi Imperfecti are divided into two families, Sporobolomycetacea (e.g., genera Sporoholomyces, Bullera) and Cryptococcaceae (e.g., genus Candida). Of particular interest to the present invention are species within the genera Pichia, Kluyveromyces, Saccharomyces, Schizosaccharomyces, and Candida. Of particular interest is the Pichia species P. pastoris. Detailed information and protocols dealing with P. pastoris may also be found in Higgins and Craig (1998). Since the classification of yeast may change in the future, for the purposes of this invention, yeast shall be defined as described in Skinner et al. In addition to the foregoing, those of ordinary skill in the art are presumably familiar with the biology of yeast and the manipulation of yeast genetics. See, for example, Bacila et al.; Rose and Harrison; Strathern et al.; herein incorporated by reference.

The nucleotide sequences of the present invention may be useful, in one embodiment of the invention, for producing biologically active mature heterologous proteins of interest in a host cell when operably linked to a promoter. In this manner, the nucleotide sequences encoding the hybrid precursor polypeptides of the invention are provided in expression cassettes for introduction into a host cell. These expression cassettes will comprise a transcriptional initiation region linked to the nucleotide sequence encoding the hybrid precursor polypeptide. Such an expression cassette is provided with a plurality of restriction sites for insertion of the nucleotide sequence to be under the transcriptional regulation of the regulatory regions. The expression cassette may additionally contain selectable marker genes.

Such an expression cassette comprises in the 5′ to 3′ direction and operably linked a host cell-recognized transcription and translation initiation region, a nucleotide coding sequence for the hybrid precursor polypeptide comprising the sequence for the mature protein of interest, and a host cell-recognized transcription and translation termination region. By “operably linked” is intended expression of the coding sequence for the hybrid precursor polypeptide is under the regulatory control of the host cell-recognized transcription and translation initiation and termination regions.

By “host cell-recognized transcription and translation initiation and termination regions” is intended regulatory regions that flank a coding sequence, in this case the nucleotide sequence encoding the hybrid polypeptide sequence, and control transcription and translation of the coding sequence in a host cell. The transcription initiation region, the promoter, provides a binding site for RNA polymerase to initiate downstream (3′) translation of the coding sequence. The promoter may be a constitutive or inducible promoter, and may be native or analogous or foreign or heterologous to the specific host. Additionally, the promoter may be the natural sequence or alternatively a synthetic sequence. By foreign is intended that the transcription initiation region is not found in the native host cell of interest into which the transcription initiation region is introduced.

The expression cassettes of the present invention can be ligated into a replicon (e.g., plasmid, cosmid, virus, mini-chromosome), thus forming an expression vector that is capable of autonomous DNA replication in vivo. Preferably the replicon will be a plasmid. Such a plasmid expression vector will be maintained in one or more replication systems, preferably two replications systems, that allow for stable maintenance within a yeast host cell for expression purposes, and within a prokaryotic host for cloning purposes. Examples of such yeast-bacteria shuttle vectors include Yep24 (Botstein el al. (1979) Gene 8:17-24; pC1/1 (Brake et al. (1984) Proc. Natl. Acad. Sci. USA 81:4642-4646), and Yrp17 (Stnichomb el al. (1982)J. Mol. Biol. 158:157).

Additionally, a plasmid expression vector may be a high or low copy number plasmid, the copy number generally ranging from about 1 to about 200. With high copy number yeast vectors, there will generally be at least 10, preferably at least 20, and usually not exceeding about 150 copies in a single host. Depending upon the heterologous protein selected, either a high or low copy number vector may be desirable, depending upon the effect of the vector and the foreign protein on the host. See, for example, Brake et al. (1984). DNA constructs of the present invention can also be integrated into the yeast genome by an integrating vector. Examples of such vectors are known in the art. See, for example, Botstein et al. (1979).

Zeocin™ is a member of the bleomycin/phleomycin family of antibiotics isolated from Streptomyces. Antibiotics in this family are broad spectrum antibiotics that act as strong anti-bacterial and anti-tumor drugs. They show strong toxicity against bacteria, fungi (including yeast), plants, and mammalian cells (Baron et al., 1992; Drocourt et al., 1990; Mulsant et al., 1988; Perez et al. 1989).

The Zeocin™ resistance protein has been isolated and characterized (Calmels et al., 1991; Drocourt et al., 1990). This protein, the product of the Sh ble gene (Streptoalloteichus hindustanus bleomycin gene), is a 13.7 kDa protein that binds Zeocin™ and inhibits its DNA strand cleavage activity. Expression of this protein in eukaryotic and prokaryotic hosts confers resistance to Zeocin™.

II. Method of the Invention

The invention includes, in one aspect, a method of quantitatively determining the relative copy number of a gene of interest introduced recombinantly into a cell. A fast, highly reliable, non-radioactive procedure has been developed for the identification of host cells containing multiple copies of genes of interest. Considered below are the steps in practicing the invention.

A. Host Cell

Exemplary host cells include prokaryotic and eukaryotic cells, and more particularly, mammalian, yeast and insect cells. The host chosen for transformation and propagation will preferably be a yeast. The yeast used in one embodiment of the method of the present invention are species within the genera Pichia. Of particular interest is the Pichia species P. pastoris. One exemplary P. pastoris strain, X-33, is used as described in Examples 1 and 2.

P. pastoris is capable of metabolizing methanol as its sole carbon source by inducing the production of alcohol oxidase (Cregg, 1993). Most P. pastoris expression strains have one or more auxotrophic mutations which allow for selection of expression vectors containing the appropriate selectable marker gene upon transformation. Prior to transformation, these strains grow on complex media but require supplementation with the appropriate nutrient(s) for growth on minimal media. P. pastoris strains of the present invention include those strains that grow on methanol at the wild-type rate (Mut⁺), and also those which vary with regard to their ability to utilize methanol because of deletions in one or both AOX genes. Also contemplated are protease-deficient strains that can be effective in reducing degradation of foreign proteins (Brierley, 1998; and White et al., 1995).

The selection of suitable yeast and other host cells for the practice of the present invention is within the skill of the art. Yeast and other microorganisms are generally available from a variety of sources, including the Yeast Genetic Stock Center, Department of Biophysics and Medical Physics, University of California (Berkeley, Calif.); the American Type Culture Collection (Manassas, Vir.); Northern Regional Research Laboratories (Peoria, Ill.); and vendors such as Invitrogen (San Diego, Calif.).

B. Expression Vector

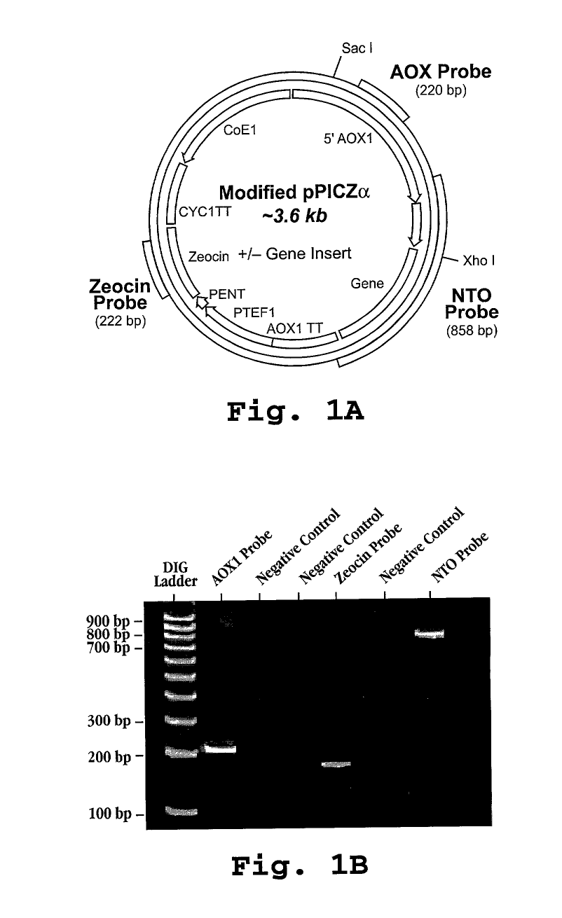

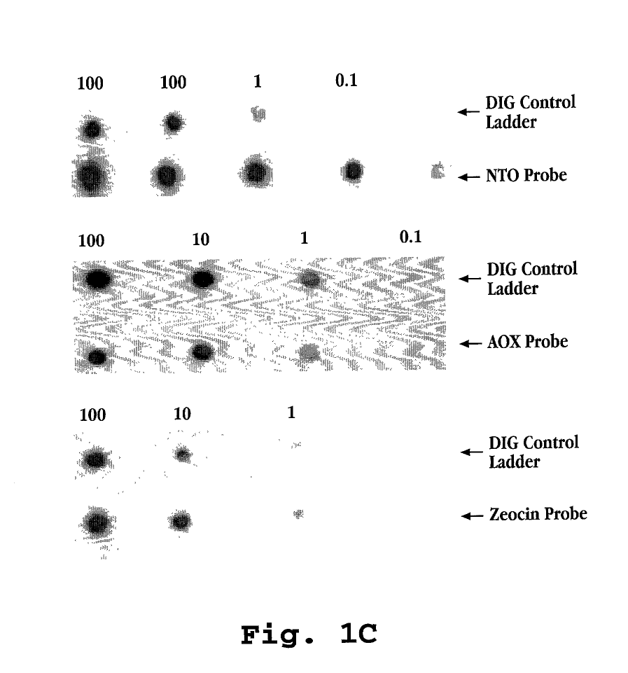

Expression vectors for use in the present invention comprise a chimeric gene (or expression cassette), designed for operation in a host cell, with companion sequences upstream and downstream from the expression cassette. The companion sequences will be of plasmid or viral origin and provide necessary characteristics to the vector to permit the vectors to move DNA from bacteria to the desired host. Suitable transformation vectors are described in co-owned U.S. application Ser. No. 60/288,206, filed May 2, 2001, which is expressly incorporated by reference in its entirety herein. Suitable components of the expression plasmid, including a trancription and translation initiator, a signal sequence, a coding sequence for the gene of interest, and suitable transcription and translation terminators are also discussed in U.S. application Ser. No. 60/288,206, referred to above. One exemplary plasmid is the modified pPICZα plasmid illustrated in FIG. 1A.

i. Selectable Markers

Selectable markers which may be included in the expression vector include the biosynthetic pathway genes HIS4 from either P. pastoris or S. cerevisiae, ARG4 from S. cerevisiae, and the Sh ble gene from Streptoalloteichus hindustanus which confers resitance to the bleomycin-related drug Zeocin (Cregg et al., 1985; Cregg and Madden, 1989; and Higgins, et aL, 1998). A more recently developed set of biosynthetic markers includes the P. pastoris ADE1 (PR-amidoimidazolesuccinocarboxamide synthase), ARG4 (argininosuccinate lyase), and URA3 (orotidine 5′-phosphate decarboxylase) genes. These markers, in one embodiment of the invention, are utilized to produce probes for hybridizing to DNA to determine the copy number of transformed cells as described below.

The pPICZα vector illustrated in FIG. 1 contains the Sh ble gene, thus conferring Zeocin resistance to the P. pastoris host into which it is transformed.

ii. Construction of Expression Vector

In preparing the expression cassette, the various nucleotide sequence fragments may be manipulated, so as to provide for the sequences in the proper orientation and, as appropriate, in the proper reading frame. Toward this end, adapters or linkers may be employed to join the nucleotide fragments or other manipulations may be involved to provide for convenient restriction sites, removal of superfluous nucleotides, removal of restriction sites, or the like. For this purpose, in vitro mutagenesis, primer repair, restriction, annealing, resubstitutions, e.g., transitions and transversions, may be involved. See particularly Sambrook et al. (1989).

The expression cassettes of the present invention can be ligated into a replicon (e.g., plasmid, cosmid, virus, mini-chromosome), thus forming an expression vector that is capable of autonomous DNA replication in vivo. Preferably the replicon will be a plasmid. Such a plasmid expression vector will be maintained in one or more replication systems, preferably two replications systems, that allow for stable maintenance within a prokaryotic host for cloning purposes and integration within a host cell for expression purposes.

Additionally, a plasmid expression vector may be integrated as a high or low copy number plasmid. A strain that contains multiple integrated copies of an expression cassette may yield, as described above, more heterologous protein than single copy strains (Clare, et al., 1991).

C. Transformation of Host Cells

Host cells are transformed with expression constructs described above using a variety of standard techniques including, but not limited to, electroporation, microparticle bombardment, spheroplast generation methods, or whole cell methods such as those involving lithium chloride and polyethylene glycol (Cregg et al., 1985; Liu et al., 1992; Waterham et al., 1996; and Cregg and Russell, 1998).

D. Propagating Transformed Cells and Isolating DNA

Transformants are grown in an appropriate nutrient medium, and, where appropriate, maintained under selective pressure to insure retention of endogenous DNA. Where expression is inducible, growth can be permitted of the host to yield a high density of cells, and then expression is induced.

Plasmid and genomic DNA may be isolated by any manner known to those of skill in the art. See, for example, Sambrook, et al., 1989. The DNA may be digested with restriction enzymes as required, separated on a gel, and transferred to nitrocellulose as described in Southern, 1975. In one embodiment, the DNA is transferred to a charged nylon membrane and analyzed with a Slot Blot. Exemplary procedures for isolating DNA, performing restriction enzyme digestion, and DNA transfers are described in Example 2.

E. Determining Copy Number

The copy number of the gene of interest in the host cell may be quantitated by determining the ratio of the control nucleic acid segment to the region of native genomic DNA in the isolated DNA. An exemplary method for determining the copy number is described in Example 2.

In one embodiment, the control nucleic acid segment is a portion of the AOX1 gene. Following transfer of the DNA to a membrane, a labeled probe capable of hybridizing with the control nucleic acid segment, the native genomic DNA, or both is contacted with the transferred DNA. Exemplary hybridization conditions are described in Example 2. The detection system utilized to quantitate the hybridized probes will depend on the label attached to the probe. In one embodiment, the probes detect less than 100 picograms of DNA. In another embodiment, the probes detect less than 10 picograms of DNA. Preferably the probes detect between 5 and 10 picograms, more preferably between 1 and 5 picograms, and even more preferably less than 1 picogram of probe-specific DNA.

Exemplary detection systems are known to those of skill in the art, and include the light emitting luciferase detection system, the NADH light adsorption detection system, fluorescence emissions and mass spectrometry as described in U.S. Pat. No. 6,270,974, which is incorporated by reference herein in its entirety. An exemplary chemiluminescent detection method is described in Example 2 below.

In another embodiment, the control nucleic acid segment and the region of native genomic DNA are amplified to generate two products. This invention is used in conjunction with the amplification of a target polynucleotide by by any method. These amplification techniques include polymerase chain reaction (PCR), ligase chain reaction (LCR), gap LCR, transcription mediated amplification (TAM), nucleic acid sequence based amplification (NASBA), and strand displacement amplification (SDA), as described in U.S. Pat. No. 6,280,930, which is incorporated by reference herein in its entirety. PCR is of particular interest. PCR is described in many references, such as Innis et at, 1989; Sambrook et al., 1989, and the like. The quantity of the amplified products generated are then determined by methods known to those of skill in the art.

In yet another embodiment, the isolated DNA is transferred to first and second regions on a substrate. On the first substrate region, a first labeled nucleic acid probe is hybridized to a region of DNA present in the host cell genomic DNA, but not present in the construct used to transform the host cell. On the second substrate region, a second labeled nucleic acid probe is hybridized to a region of DNA present in the construct, but not present in the genomic DNA of the host cell prior to transformation with the construct. The signal intensity of the first and second labeled probes is then determined as described above.

The isolated DNA is preferably transferred to the substrate regions in known quantities. In one embodiment, the quantity of DNA transferred to each region is equal. In another embodiment, the substrate contains two or more regions, and the DNA is diluted by a known amount prior to being transferred to each region. One method for making ordered arrays of DNA on a membrane is a “dot blot” approach. In this method, a vacuum manifold transfers a plurality, e.g., 96, aqueous samples of DNA from 3 millimeter diameter wells to a porous membrane. A common variant of this procedure is a “slot-blot” method in which the wells have highly-elongated oval shapes. Other methods of making ordered arrays of DNA known to those of skill in the art may be employed in the present invention. See, for example, U.S. Pat. No. 6,312,960, which is incorporated by reference herein. Exemplary substrates onto which DNA may be immobilized include, without limitation, nylon membranes, nitrocellulose, polypropylene, polystyrene, vinyl, other plastics and glass.

III. Kit

As a matter of convenience, the reagents employed in the present invention can be provided in a kit packaged combination with predetermined amounts of reagents for use in determining and/or quantitating gene copy numbers. For example, a kit can comprise in packaged combination with other reagents any or all of primers or probes described depending on need. Generally, it is desirable to include the requisite number of probes and/or primers to afford determination of the copy number as referred to above. The oligonucleotide probes can be labelled or bound to a support or can be provided with groups that permit the probe or primer to be subsequently labelled or bound to a support. The kit can further include in the packaged combination buffers, developing systems for the selected label, other necessary enzymes, nucleoside triphosphates and the like.

Additionally, the kit may optionally contain a denaturation solution, a hybridization buffer, a wash solution and a substrate. It is also envisioned that the kit contain a internal calibration standard.

IV. Examples

The following examples further illustrate the invention described herein and are in no way intended to limit the scope of the invention.

### EXAMPLE 1

### Cloning the Gene of Interest into pPICZ A, B, and C

pPICZ A, B, and C vectors, 3.3 kb in size, are used to express recombinant proteins in Pichia pastoris. The gene of interest is cloned into the multiple cloning site of pPICZ using standard molecular biology protocols. Recombinant proteins are expressed as fusions to a C-terminal peptide containing the c-myc epitope and a polyhistidine (6×His) tag. The vector allowed high-level, methanol inducible expression of the gene of interest in Pichia, and is used in Pichia strains X-33, GS115, SMD1168H, and KM71 H. pPICZ contains the following elements: 5′ fragment containing the AOX1 promoter for tightly regulated, methanol-induced expression of the gene of interest and target plasmid integration to the AOX1 locus (Ellis et al, 1985; Koutz et al., 1989; Tschopp et al., 1987); Zeocin™ resistance gene for selection in both E. coli and Pichia (Baron et al., 1992; Drocourt et al., 1990); C-terminal peptide containing the c-myc epitope and a polyhistidine (6×His) tag for detection and purification of a recombinant fusion protein (if desired); three reading frames to facilitate in-frame cloning with the C-terminal peptide.

pPICZ vectors are propagated in either TOP10, JM109, or DH5α. Chemical and electroporation transformation methods are well know in the art. See, e.g. Sambrook et al., 1989; and Ausubel et al., 1994.

### EXAMPLE 2

### Determination of Copy Number using Diqoxigenin-labeled Probes

The pPICZ expression vector features a promoter and other 5′ and 3′ sequences derived from the alcohol oxidase gene I (AOX1) from P. pastoris, and drug resistance to Zeocin™. Two sets of PCR primers were designed to generate digoxigenin-labeled probes, one specific for the AOX1 region and the other for the Zeocin™ region of the vector. These probes were consistent in detecting less than or equal to 10 picograms of probe-specific DNA in a Southern blot using chemiluminescence detection. Southern blots of SacI-cut P. pastoris DNA from the untransformed clone revealed a single 9 kb band that hybridized with the AOX1 probe but not with the Zeocin™ probe. Both sets of probes, however, hybridized with the 9 kb band and a lower band, corresponding to the linearized cassette length, from SacI-cut P. pastoris recombinant DNA.

Several of the clones showed a vector-specific lower band with greater hybridization signal intensity than the AOX1-specific band, clearly indicating that these clones contained multiple copies of the expression cassette. Some clones also gave larger bands that were consistent in size witht he loss of one or more SacI sites—presumably by exonucleolytic trimming in vivo prior to integration. Densitometer scanning of the blot and quantitative slot-blot hybridization analysis was used to determine the precise copy numbers of the clones. These results were in accordance with the sizes of Zeocin™-specific bands generated from partial Sac I-cut recombinant DNA that corresponds to tandem head-to-tail arrays at the AOX1 locus. Thus, selection of multicopy clones using a non-radioactive technique has safety and environmental benefits as well as high-yield production advantages.

A. Materials

All P.Pastoris yeast expression reagents were purchased from Invitrogen (Carlsbad, Calif.). Genomic Isolation materials were purchased from CPG Inc. (Lincoln Park, N.J.). Plasmid Isolation materials were purchased from Eppendorf (Westbury, N.J.). Sac I restriction endonuclease and reaction buffer was purchased from Roche (Indianapolis, Ind.).

The Zeocin and AOX region primer sets were synthesized by Life Technologies (Gaithersburg, Md.). PCR reagents, except primer sets, were purchased from Perkin-Elmer/Roche (Branchburg, N.J.). Microcon-100 and Micropure-EZ filter units were purchased from Millipore Corporation (Bedford, Mass.). Dig-11-dUTP and DIG-Labeled Control DNA was purchased from Roche. Agarose powder and ethidium bromide was purchased from Life Technologies. DNA Molecular Weight Marker's II, III, and VII were purchased from Roche. 6X Gel Loading Buffer was purchased from Invitrogen. Positively Charged Nylon Membranes, DIG Hybridization Buffer, CSPD (Disodium 3-(4-methoxyspiro {1,2-dioxetane-3,2′-(5′-chloro)tricyclo[3.3.1.1^{3,7}]decan}-4-yl) phenyl phosphate), Lumi- Film and DIG Wash and Block Buffer Set were purchased from Boehringer Mannheim. 20X SSC and 10% SDS was purchased from GibcoBRL. Developer and Fixer solutions were purchased from Kodak.

B. Procedure

1. DIG-Labeling of Probes

The labeling of the probes was performed by doing two consecutive PCR reactions. All PCR reagents, except the Zeocin and AOX primer pairs, were purchased from Perkin Elmer. The first round PCR was performed in a 50 μL volume consisting of 10 pmoles of respective 5′ and 3′ primers identified in Table 2 below (NTO [SEQ ID NOS: 5 and 6], AOX1 [SEQ ID NOS: 1 and 2] or Zeocin [SEQ ID NOS: 3 and 4]), 2.5 mM MgCl₂, 125 μM of dATP, 125 μM dTTP, 125 μM dCTP, 125 μM dGTP, 1X PCR Buffer II (50 mM KCl, 10 mM Tris-HCl, pH 8.3), 5U of Amplitaq Enzyme, and 100 ng template (For the Zeocin and AOX probe a pPiczαB plasmid template was used, for the NTO probe a 100 ng NTO plasmid template was used. NTO and NPR plasmids are variants of the pPICZα vector—both plasmids contain the AOX1 and Zeocin genes and synthetic genes of interest. See U.S. Pat. No. 6,204,022, which is incorporated by reference in its entirety herein.) The PCR amplification was done for 30 cycles (Zeocin Probe: 1 minute at 95° C., 1 minutes at 58° C., and 1 minute at 72° C. 5′/3′ NTO Probe: 1 minute at 95° C., 1 minutes at 54° C., and 1 minute at 72° C. 11/12 AOX Probe: 1 minute at 95° C., 1 minutes at 50° C., and 1 minute at 72° C.) with a final extension of 7 minutes at 72° C. on a Perkin Elmer DNA Thermal Cycler 480. After the PCR amplification the samples were cleaned and concentrated to a 20 μL volume using Micropure-EX and Microcon-100 filter units (Millipore Corporation).

The concentrated PCR product was run on a 2% Agarose Gel and spot-densitometry with the Alphalmager (Alpha Innotech Corporation) was used to determine the concentration. The second labeling PCR reaction was performed in a 50 μL volume consisting of 60 ng of clean product from the PCR round 1, 10 pmoles of respective 5′3′ primers, 2.5 mM MgCl₂, 200 μM dATP, 130 μM dTTP, 200 μM dCTP, 200 μM dGTP, 0.07 mM DIG-11-dUTP, 1X PCR Buffer II, and 5U of Amplitaq Enzyme. The PCR labeling was run under the same conditions as the amplification and was also done for 30 cycles with a final extension of 7 minutes at 72° C. on a Perkin Elmer DNA Thermal Cycler 480.

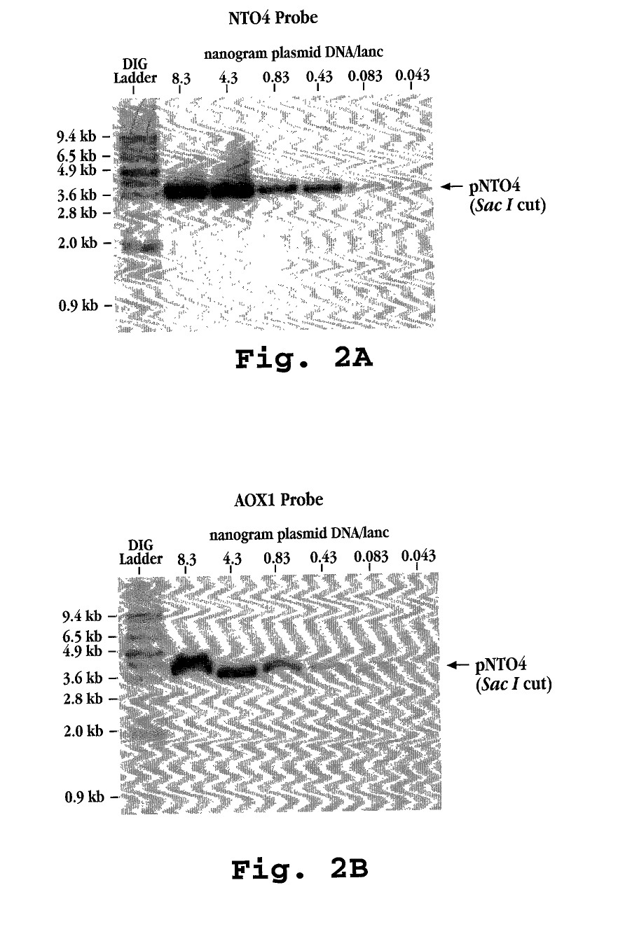

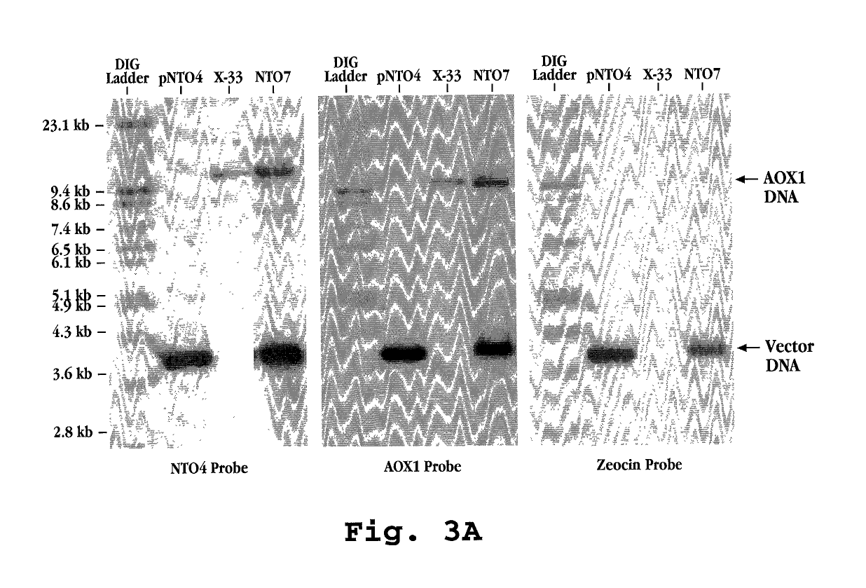

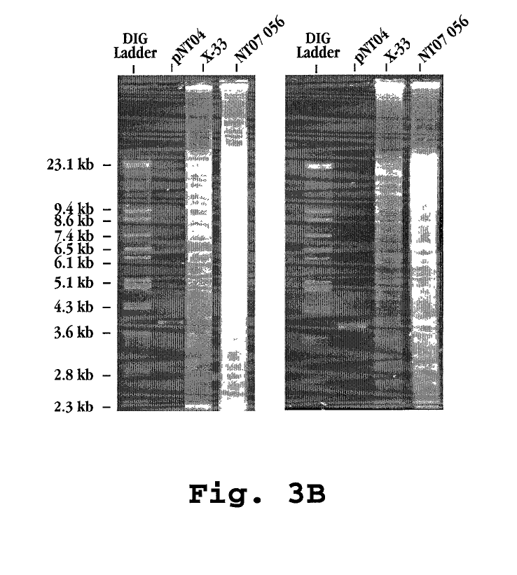

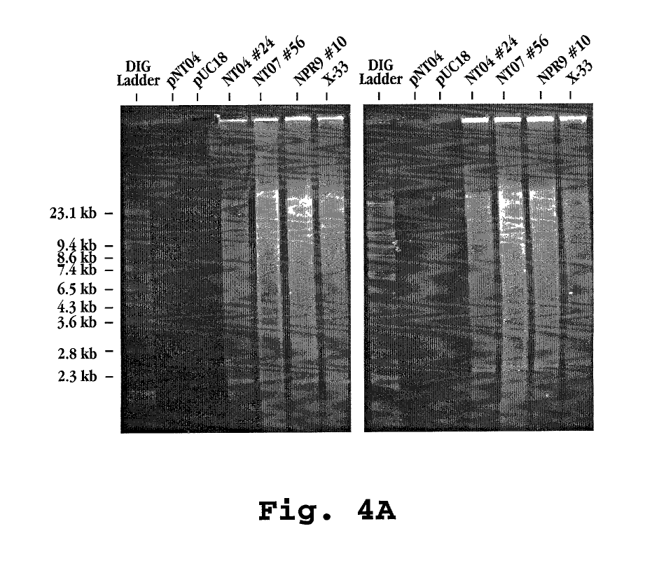

The probe concentration was determined by using spot-densitometry with the Alphalmager (Alpha Innotech Corporation). A dilution series of the probes were spotted on a positively charged nylon membrane (Roche) along with a dilution series of a DIG-Labeled Control DNA (Roche). The positively charged membrane was crosslinked using Hoefer UVC500 UV Crosslinker (Amersham Pharmacia Biotech) set at 120,000 μJ/cm². The membrane was detected following CSPD chemiluminescence system procedure provided from Roche (see under Chemilumenescent detection for further details). Using spot-densitometry with the Alphalmager (Alpha Innotech Corporation) and the dilution series of the DIG-Labeled Control DNA set as the standard the probe concentration were determined. See FIGS. 1A-1C, 2A-2C, and 3A-3C showing the use of the digoxigenin-labeled probes.

2. Genomic/Plasmid Isolation Procedures

Bacterial clones were grown in 5 mL of Low Salt LB (LSLB) Media with Zeocin [25 μg/mL] (Invitrogen) for 8 hours at 37° C./250 RPM in Orbital Shaker Model 4518 (Forma Scientific). The first day growth was inoculated into 40 mL of fresh LSLB with Zeocin and growth was continued at 37° C./250RPM in Orbital Shaker Model 4518 (Forma Scientific). After a 16-hour growth, plasmid isolations were performed using the procedure outlined in the Perfect Prep Plasmid Midi Kit (Eppendorf). The plasmid yields were determined by taking the 260/280/320 absorbance readings using Ultrospec 3000pro UV/Visible Spectrophotometer (Amersham Pharmacia Biotech). Genomic Isolations were performed after a one-day growth in 10 mL of Yeast Peptone Dextrose (YPD) media with Zeocin (100 μg/mL) at 37° C./250RPM in Orbital Shaker Model 4520 (Forma Scientific). The Genomic Isolation procedure was performed following the protocol in the DNA-Purem Yeast Genomic Kit (CPG). The yields were determined by taking the 260/280/320 readings using Ultrospec 3000pro UV/Visible Spectrophotometer (Amersham Pharmacia Biotech).

3. SACI Endonuclease Restriction Enzyme Cuts

All restriction enzyme cuts were performed in 20 μL volumes. 10 g cuts of genomic DNA was cut using SACI endonuclease restriction enzyme (1 U/ g) and Buffer A (Roche) incubated at 37° C. overnight in a Perkin Elmer DNA Thermal Cycler 480. For Plasmid DNA 2 μg cuts were performed with SACI Endonuclease Restriction Enzyme and Buffer A (Roche) for 2 hours at 37° C. in a Perkin Elmer DNA Thermal Cycler 480. The Partial Restriction Enzyme Cuts were performed using a 0.03 U/μg SACI Enzyme concentration on 10 μg of Genomic DNA sample for 15 minutes at 37° C. in a Perkin Elmer DNA Thermal Cycler 480. All the enzyme cuts were stopped by adding 4 μL of 6X Loading Buffer (Invitrogen) and cooling samples down to 2-8° C.

4. Gel Electrophoresis and Southern Blot Transfer

8.3 ng of SACI cut plasmid DNA or 10 μg of SACI cut genomic DNA were loaded onto a 0.7% agarose gel and run for 360-450 volt hours. 5 μL of DNA Molecular Weight Marker's II, III, and VII (Roche Biomolecular) were run as ladders on each gel. The gels were stained in EtBr and the DNA visualized using an Alphalmager (Alpha Innotech Corporation). Prior to transfer a denaturing step (0.5M NaOH; 1.5M NaCl solution) and a neutralizing step (0.5M Tris-HCl (pH 8.0); 1.5M NaCl solution) were performed to increase transfer efficiency. The southern transfers were performed using capillary action in 10× SSC buffer onto a positively charged membrane. After the transfer the membranes were crosslinked using Hoefer UVC500 UV Crosslinker (Amersham Pharmacia Biotech) set at 120,000 μj/cm².

5. Slot Blot

Slot Blot analysis was performed using Bio-Dot® (Bio-Rad) Slot Blot apparatus with a Vac® V-500 (Buchi) vacuum pump. The positively charged nylon membrane was pre-soaked in sterile roH₂O and 10× SSC prior to setting up the apparatus. 10 μg of chromosomal DNA was denatured for 15 minutes in 1 M NaOH. The samples were neutralized with 1 M HCl and diluted with 10× SSC prior to loading into the appropriate slots. Each slot was rinsed with 10× SSC prior to loading samples and rinsed with 2× SSC after the samples had run through. The membrane was then crosslinked using the Hoefer UVC500 UV Crosslinker (Amersham Pharmacia Biotech) set at 120,000 μj/cm².

6. Probe Hybridization Chemiluminescent Detection

Cross-linked membranes were pre-hybridized with 8 mL of DIG Hybridization Buffer (Roche Biomolecular) and incubated at 42° C. in a Hoefer HB400 Hybridization Oven (Amersham Pharmicia Biotech). Following pre-hybridization, 100-150ng of Probe was boiled, chilled, and then loaded into the hybridization tubes. The hybridization was allowed to run 14-18 hours at42° C. in Hoefer HB400 Hybridization Oven (Amersham Pharmicia Biotech). Following Hybridization stringency washes were performed with 2× SSC+0.1% SDS (All probes: 2×25 mL washes at 42° C.) and 0.1× SSC+0.1% SDS (2×25 mL washes at 50° C. [AOX and Zeocin Probes] or 60° C. [NTO probe]).

7. Chemiluminescent Detection

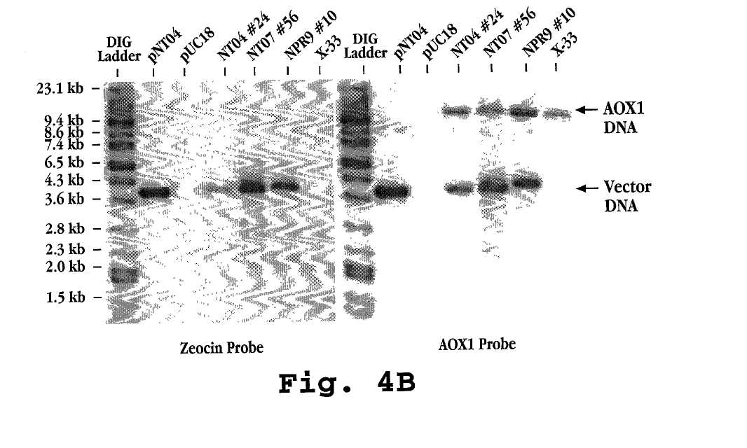

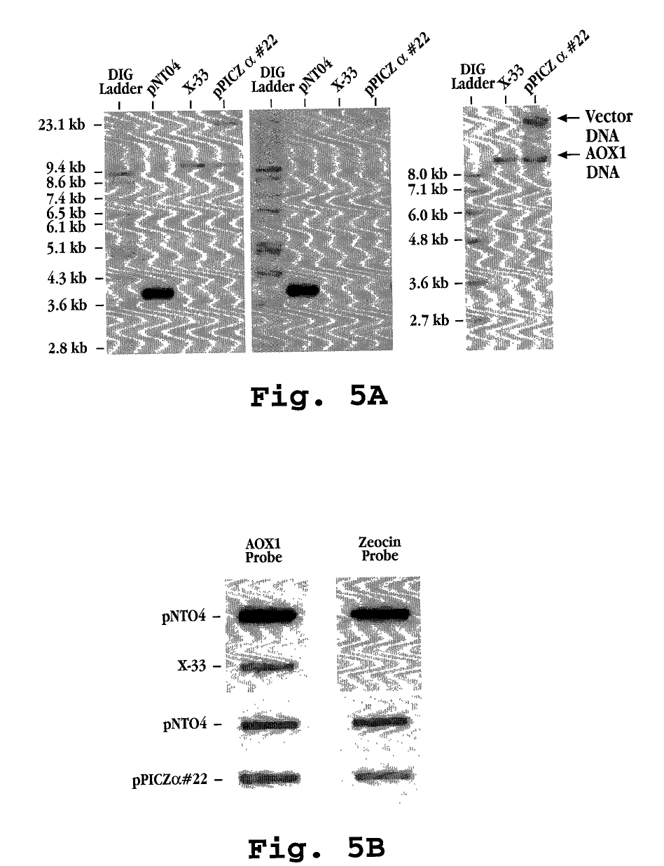

The chemiluminescent detection protocol used is as outlined in CSPD® product insert (Roche Biomolecular) with the exception of increasing in the blocking time to 1 hour and adding a third 15 minute wash after incubating with the anti-Dig antibody Solution. The buffers used are as follows: Washing Buffer (3% Tween in 0.1 M Maelic Acid), Detection Buffer (0.1 M Tris-HCl, 0.1 M NaCl, pH 9.5), Antibody Solution (1:10,000 dilution of Anti-Digoxigenin-AP [Roche]), and Blocking Solution (Roche). Blots were developed in an Autoradiography Cassette (Fisher Biotech) using Lumi-Film Chemillumenescent Film (Roche) with Kodak GBX Developer/Fixer. Pictures of films and spot-densitometry analysis were performed using the Alphalmager (Alpha Innotech Corporation, San Leandro, Calif.). FIGS. 4A-4B and 5-5B show the ratio of the 10 kb and 4 kb band intensities and the high molecular weigh vector-specific DNA band.

Densitometric analysis in Table I below shows that the ratios of the intensities of the vector-specific DNA to the AOX-specific DNA can be used to give an estimated number of vector copies inserted into the Pichia pastoris genome of the recombinant.

TABLE 1
Densitometric Analysis to Determine Gene Copy Number

| | NTO4 #24 | NTO7 #56 | NPR9 #10 |
|---|---|---|---|
| AOX1 | | | |
| Area Under Peak | 2072 | 1879 | 2872 |
| Peak % of Lane | 42.7 | 26.7 | 43.2 |
| Vector | | | |
| Area Under Peak | 2776 | 5166 | 3774 |
| Peak % of Lane | 57.3 | 73.3 | 56.8 |
| Ratio (Vector/AOX1) | 1.34 | 2.75 | 1.31 |
| Estimated Copy | 1 | 3 | 1 |
| Number | | | |

TABLE 2
Gene Copy Number Primers

| | | | PCR |
|---|---|---|---|
| Primer Set | | | Product |
| (Description) | Primer Name | Primer Sequence | Length |
| AOX1 Probe | N0011 | 5′-GCG AGG TTC ATG TTT GTT TAT TTC- | 220 bp |
| | | 3′ (SEQ ID NO: 1) | |
| | N0012 | 5′-ACT GGC CGT TAG CAT TTC A-3′ (SEQ | |
| | | ID NO: 2) | |
| Zeocin Probe | N0009 | 5′-GCC GGA GCG GTC GAG TTC TGG-3′ | 222 bp |
| | | (SEQ ID NO: 3) | |
| | N0010 | 5′-GGA GGC GTC CCG GAA GTT CGT G- | |
| | | 3′ (SEQ ID NO: 4) | |
| NTO4 Probe | 5′-AOX1 | 5′- GAC TGG TTC CAA TTG ACA AGC-3′ | 858 bp |
| | | (SEQ ID NO: 5) | |
| | 3′-AOX1 | 5′-GCA AAT GGC ATT CTG ACA TCC-3′ | |
| | | (SEQ ID NO: 6) | |

TABLE 3
Densitometric analysis of blots from FIGS. 5A-5B

| | Densitometry | Standardized | % AOX1 | |
|---|---|---|---|---|
| | Values | Ratio | Ratio | Copy Number |
| pNTO4 | 3417 | 1 | — | — |
| (AOX1) | | | | |
| pNTO4 | 2987 | 1 | 100% | |
| (Vector) | | | | |
| pPICZα #22 | 2361 | 0.69 | — | — |
| (AOX1) | | | | |
| pPICAα #22 | 1767 | 0.59 | 85.5% | 6 |
| (Vector) | | | | |

Table 3 shows the densitometric analysis of duplicate slot blots hybridized with either the AOX1 probe or the Zeocin probe. The results show the pPICZα#22 recombinant clone to have an estimated 6 cassette copies of vector DNA inserted into the Pichia pastoris genome. Note that the pPICZα#22 DNA densitometry values from each blot was standardized to that of the plasmid pNTO4 control DNA from the same blot (e.g. pPICZα#22 value/pNTO4 value). The standardized values were then used to determine the percent AOX1 ratios that represent the percentage of the AOX1 probed intensity belonging to the pPICZα#22 vector DNA. Standardized ratio is equal to (pPICZα#22 Densitometry Values)/(pNTO4 Densitometry Values). % AOX1 ratio is equal to (Vector Standardized Value)/(AOX1 Standardized Value). Estimated copy number is equal to (100/(100-% of AOX1 Ratio))-1.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the invention has been described with respect to particular embodiments, it will be apparent to those skilled in the art that various changes and modifications can be made without departing from the invention.

It is claimed:

1. A method of quantitatively determining the relative copy number of a gene of interest introduced recombinantly into a cell; comprising
a) propagating, in a suitable culture medium, cells that are transformed with a chimeric gene construct containing and capable of expressing the gene introduced into the cells, where said construct includes;
(i) a control nucleic acid segment having a region of DNA capable of hybridizing with a region of native genomic DNA associated with the cell; and
(ii) a second nucleic acid region that contains said gene of interest;
b) isolating DNA from said cells; and
c) determining the ratio of said control nucleic acid segment to said region of native genomic DNA in the isolated DNA as a quantitative measure of the relative copy number of said gene in said cell.

2. The method of claim 1, wherein determining said ratio comprises;
a) separating said isolated DNA;
b) hybridizing a labeled nucleic acid probe to said control nucleic acid segment and to said region of native genomic DNA; and
c) comparing signal intensity of said label hybridized to said control nucleic acid segment with said region of native genomic DNA.

3. The method of claim 1, wherein said determining comprises;
a) amplifying said control nucleic acid segment and said region of native genomic DNA to generate two PCR products; and
b) comparing signal intensity of said PCR products.

4. The method of claim 3, wherein said amplifying is performed using labeled PCR primers.

5. The method of claim 3, wherein said PCR products are separated and a labeled nucleic acid probe is hybridized to said products.

6. The method of claim 1, wherein determining said ratio comprises;
a) transferring said isolated DNA to first and second regions on a substrate;
b) on said first substrate region, hybridizing a first labeled nucleic acid probe to a region of DNA present in said genomic DNA, but not present in said construct;
c) on said second substrate region, hybridizing a second labeled nucleic acid probe to a region of DNA present in said construct, but not present in said genomic DNA; and
d) comparing signal intensity of said first and second labeled probes.

7. The method of claim 6, wherein said isolated DNA is transferred to said first and second substrate regions in equal quantities.

8. The method of claim 6, wherein said substrate is a nylon membrane.

9. The method of claim 1, wherein said chimeric gene construct is integrated into said cell genome, and determining said ratio is used to confirm stability or copy number of said gene of interest.

10. The method of claim 1, wherein less than or equal to 100 picograms of said control nucleic acid segment or said region of native genomic DNA is detected.

11. The method of claim 1, wherein less than or equal to 100 picograms of said control nucleic acid segment or said region of native genomic DNA is detected.

12. The method of claim 1, wherein said cells are yeast cells.

13. The method of claim 12, wherein said cells are P. pastoris cells.

14. The method of claim 2, wherein said label is digoxigenin.
